(19) United States
(12) Patent Application Publication
(10) Pub. No.: US 2013/0321168 A1
(43) Pub. Date: Dec. 5, 2013

(54) SURVIVAL AND LOCATION ENHANCEMENT GARMENT AND HEADGEAR

(71) Applicants: Dennis Mahony, Coogee (NSW) (AU); Adrian Bruce, Killarney Heights (NSW) (AU); Michael Batty, North Epping (NSW) (AU); Valerie Kuo, Carlingford (NSW) (AU); Andrew Wyatt, Chatswood (NSW) (AU)

(72) Inventors: Dennis Mahony, Coogee (NSW) (AU); Adrian Bruce, Killarney Heights (NSW) (AU); Michael Batty, North Epping (NSW) (AU); Valerie Kuo, Carlingford (NSW) (AU); Andrew Wyatt, Chatswood (NSW) (AU)

(73) Assignee: JOELMAR PTY LTD., Mascot (NSW) (AU)

(21) Appl. No.: 13/982,298

(22) PCT Filed: Feb. 21, 2012
(86) PCT No.: PCT/AU2012/000155

§ 371 Date: Jul. 29, 2013

(30) Foreign Application Priority Data
Feb. 22, 2011 (AU) 2011900594

(51) Int. Cl. A61B 5/00

(52) U.S. Cl. CPC A61B 5/002
USPC 340/870.09; 340/870.07

(57) ABSTRACT

The present invention provides system of active uniform and base station for sensing an aspect of the wearers environment or physiology. Active uniform (1) is comprised of uniform (3), electronic sensors (40) for sensing an aspect of the wearer's environment or of the wearer's physiology and an active tag (10). The system of active uniform (1) and base station (2) is used to collect data from wearers of the at least one item of active uniform which allows an assessment of the health of the wearer where said health assessment is subsequently used to enhance the survivability of the wearer. The active tag contains various components that allow it to communicate with the base station (2) including (i) sensor interface (80) for interfacing electronic sensors (40) to the active tag, (ii) a microcontroller (70), (iii) a data store (60) including flash memory, (iv) radio frequency interface (110), (v) at least one tag antenna (120) and (vi) a battery (100) and power management unit (90). The sensors (40) and active tag (10) are adapted to be retained on or in the item of uniform (3) such that the active uniform (1) is able to be washed without removing the electronic sensors (40) or the active tag (10). Base station (2) comprises base station antenna (20), base station transceiver (320) and data processing apparatus (340) connected to the base station transceiver, which is adapted to receive and store data transmitted by the active tag of the active uniform, including at least, sensor and identification data.

### TECHNICAL FIELD

The field of the present invention relates to active uniforms including, headgear and footwear, that collect environmental and wearer data for transmission, recording and subsequent analysis by data processing apparatus such that the survivability of the wearer of the active uniform is enhanced in hazardous conditions. The invention also relates to wearable electronic devices incorporated into garments and uniforms for use in hazardous conditions. Active uniforms that enhance the survivability of the wearer would find application in military, industrial, medical and civil applications where the wearer is subject to hazardous conditions or is otherwise the subject of monitoring for signs of ill health.

### BACKGROUND ART

Hitherto, garments with associated sensors for physiological monitoring have been described, particularly for use in the medical arena. Such garments however are unsuitable for use in the military and with respect to use in hazardous conditions such as in fire fighting. Such garments are not useful as they are not robust, are difficult to wear, would otherwise be unreliable due to the operational environment and would be unlikely to enhance the survivability of the wearer.

### DISCLOSURE OF INVENTION

According to one aspect of the present invention, there is provided a system for sensing, logging and presenting physiological and/or environmental conditions of a wearer of an item of active uniform, the system comprising an active uniform and a base station; wherein the active uniform is comprised of:
(i) an item of uniform;
(ii) at least one electronic sensor for sensing an aspect of the wearer's environment or of the wearer's physiology
(iii) an active tag mounted on the item of uniform for communicating with the at least one electronic sensor, wherein the active tag comprises
   (a) sensor interface for interfacing the at least one electronic sensor to the active tag
   (b) at least one microcontroller adapted to:
      process the signals received over the sensor interface from the at least one electronic sensor into sensor data
      send and receive sensor data, and identification data which identifies the wearer, to a base station transceiver;
      send/receive and/or process instructions sent or received from the base station transceiver, including where necessary, the control of other components of the active tag;
   (c) data store for recording and storing, at least, sensor data;
   (d) radio frequency interface adapted to convert electrical signals output by the microcontroller into radio signals adapted to be received by the base station transceiver and to convert radio signals received from the base station transceiver, into electrical signals that are adapted to be received by the microcontroller
   (e) at least one tag antenna for transmitting radio signals to and from the base station transceiver;
   (f) power supply and regulation means; and wherein the base station comprises:
(i) at least one base station antenna;
(ii) a data processing apparatus connected to the transceiver, which is adapted to, at least, receive and store data transmitted by the active tag of the active uniform, including at least, sensor and identification data.

Preferably, the at least one electronic sensor and active tag are waterproof and wherein the active uniform can be laundered in conventional laundry machines without removing the at least one electronic sensor and active tag.

More preferably the microcontroller combines and processes sensor data from the plurality of different electronic sensors to arrive at a measure of something not able to be measured directly or is difficult to measure directly, by way of an electronic sensor.

Still more preferably the microcontroller combines and processes sensor data from the plurality of electronic sensors to arrive at a more accurate measurement of something able to be measured directly by way of an electronic sensor.

According to a second aspect of the invention there is provided the active uniform of the system described above.

According to a third aspect of the invention there is provided the base station of the system described above.

According to a fourth aspect of the invention there is provided, a method of increasing the survivability of the wearer of an active uniform of the present invention, the method comprising:
providing at least one item of active uniform which continuously monitors and records the environment and/or physiological functions of the wearer's data,
transmitting the data to an authenticated transceiver
receiving the data at the authenticated transceiver and communicating the data to a data processing device
processing the data to determine whether either the environmental conditions experienced by the wearer or the wearer's own physiological data indicates that the wearer should be taken out from active service, or have future duties modified, or medically treated, so as to enhance the survivability of the wearer.

### BRIEF DESCRIPTION OF THE DRAWINGS

FIG. 1 is a schematic representation of two active uniforms incorporating active tags in connection with a base station comprising, an array of antennas, a transceiver, and a data processing apparatus, as well as an exploded schematic of the components of the active tag, according to a first embodiment of the invention;

FIG. 2 is a schematic view of short range antenna portals in association with the two active uniforms according to a first embodiment of the invention;

FIG. 3 is a schematic view of longer range antenna portals according to a first embodiment of the invention;

FIG. 4 is a schematic view of the active tag components according to a first embodiment of the invention;

FIG. 5 is a diagram of the PCB construction of the active tag to produce an enclosure and a convoluted electrical conductor forming a continuous circuit around the electronics according to a first embodiment of the invention;

FIG. 6 is a schematic view of an array of microelectrogenerators embedded in uniforms according to a first embodiment of the invention;

FIG. 7 is a schematic view of an array of microelectrogenerators of FIG. 6 indicating how they can collect environmental energy, such as light, RF energy or from bending motion or other movement;

FIG. 8 is a top view of an active tag with a connector between the encapsulated circuit board and the cable with no air gaps or masking materials in the encapsulation according to a first embodiment of the invention;

FIG. 9 is a top view of an active tag with an air gap over the antenna matching components and a masking material over the battery according to a first embodiment of the invention;

FIG. 10 is a top view of an active tag with a piece of foam over the antenna matching components and a masking material over the battery according to a first embodiment of the invention;

FIG. 11 is a bottom view of an active tag with an air gap behind the antenna and a masking material over the battery according to a first embodiment of the invention;

FIG. 12 is a side view of an active tag with no air gaps, foam or masking material in the encapsulation with the sensor cables exiting the encapsulation through a connector and strain relief according to a first embodiment of the invention;

FIG. 13 is a side view of an active tag with no air gaps, foam or masking material in the encapsulation with the microcontroller programming cables exiting the encapsulation according to a first embodiment of the invention;

FIG. 14 is a side view of an active tag with an air gap over the antenna matching components, an air gap behind the antenna and a masking material over the battery according to a first embodiment of the invention;

FIG. 15 is a side view of an active tag with foam over the matching components, an air gap behind the antenna and a masking material over the battery according to a first embodiment of the invention;

FIG. 16 shows a top view of an active tag sitting on top of an air filled spacer inside a pocket closed with a loop and hook fastener according to a first embodiment of the invention;

FIG. 17 shows a side view of an active tag sitting on top of an air filled spacer inside a pocket closed with a loop and hook fastener according to a first embodiment of the invention;

FIG. 18 is a schematic view of the active tag components in accordance with the third embodiment of the first aspect of the invention;

FIG. 19 is a schematic representation of a helmet and active tag shown in connection with an antenna and reader, as well as a base station data processing apparatus according to the third embodiment of the invention;

FIG. 20 is a diagram of the pressure wave which may be produced by an explosive blast;

FIG. 21 is a schematic of the internals of an active tag with two pressure, mechanical shock and accelerometer sensors according to the third embodiment of the invention;

FIG. 22 is a schematic of the internals of an active tag according to the second embodiment of the invention;

FIG. 23 is a schematic view of a soldier wearing two active uniforms and in communications with base station according to the first embodiment of the invention;

FIG. 24 is a schematic view of an array of sensors in communication with an active tag and base station antenna according to the second embodiment of the invention;

FIG. 25 is a top view of the encapsulated temperature sensor, sewn into a small pocket on the inside of the garment according to the first or second embodiment of the invention;

FIG. 26 shows a top view of a fabric tunnel sewn across the chest on the inside of the garment with ECG electronic sensors and fabric electrodes attached to the tunnel according to the first or second embodiment of the invention;

FIG. 27 is a side view of two ECG electronic sensors positioned beneath fabric electrodes with cables running inside a fabric tunnel according to the first or second embodiment of the invention;

FIG. 28 is a top cutaway view of conducting cables arranged in a flexible and stretchable configuration through a fabric tunnel sewn onto the inside of the smart garment according to the first or second embodiment of the invention;

FIG. 29 is an end view of the fabric tunnel showing the cross section of the conducting cables through the tunnel according to the first or second embodiment of the invention;

FIG. 30 is an end view of the fabric tunnel showing the layers of the fabric electrodes, ECG electronic sensors, fabric tunnel, cables, and garment according to the first or second embodiment of the invention;

FIG. 31 is an end view of a third ECG sensor attached to a fabric electrode where the fabric electrode is sewn over the sensor to form a pocket on the inside of the uniform according to the first or second embodiment of the invention;

FIG. 32 is a front view of the garment showing two ECG sensors and the cable tunnel sewn on the inside of the garment's front panel, where the front half of a flexible strap with a fastening device is shown attached at one side of the garment, which may be of a hook and loop construction according to the first or second embodiment of the invention;

FIG. 33 is a back view of the garment showing a third ECG sensor sewn on the inside of the garment's back panel and the back half of the flexible strap attached at the side according to the first or second embodiment of the invention;

FIG. 34 is a front view of the garment with the front and back straps secured over the ECG sensors according to the first or second embodiment of the invention;

FIG. 35 shows the front view of the garment with an integrated elastic strap around the chest sensors according to the first or second embodiment of the invention;

FIG. 36 is a side view of the encapsulated temperature sensor, sewn into a small pocket on the inside of the garment according to the first or second embodiment of the invention;

FIG. 37 is a diagram of the modules in the active tag firmware;

FIG. 38 is a flow chart of the active tag firmware converting ECG signals into a heart rate;

FIG. 39 is a flow chart of the PC software communicating with the active tag via the base station transceiver and processing the logged data; and

FIG. 40 is a flow chart of the active tag firmware modules for acquiring, processing and saving sensor data.

### MODES FOR CARRYING OUT THE INVENTION

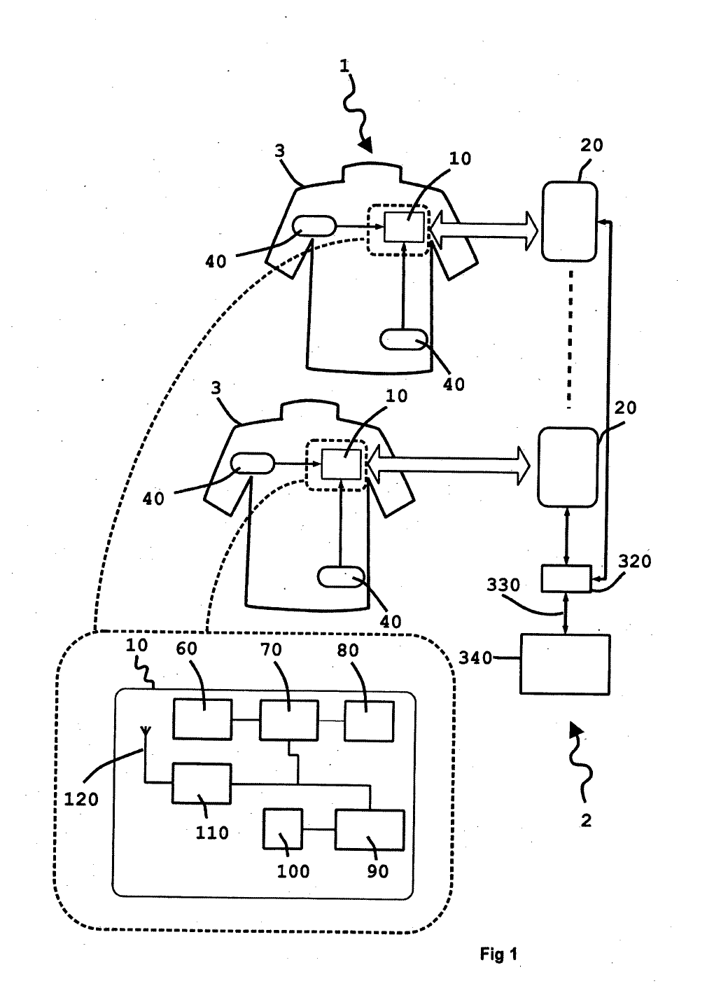

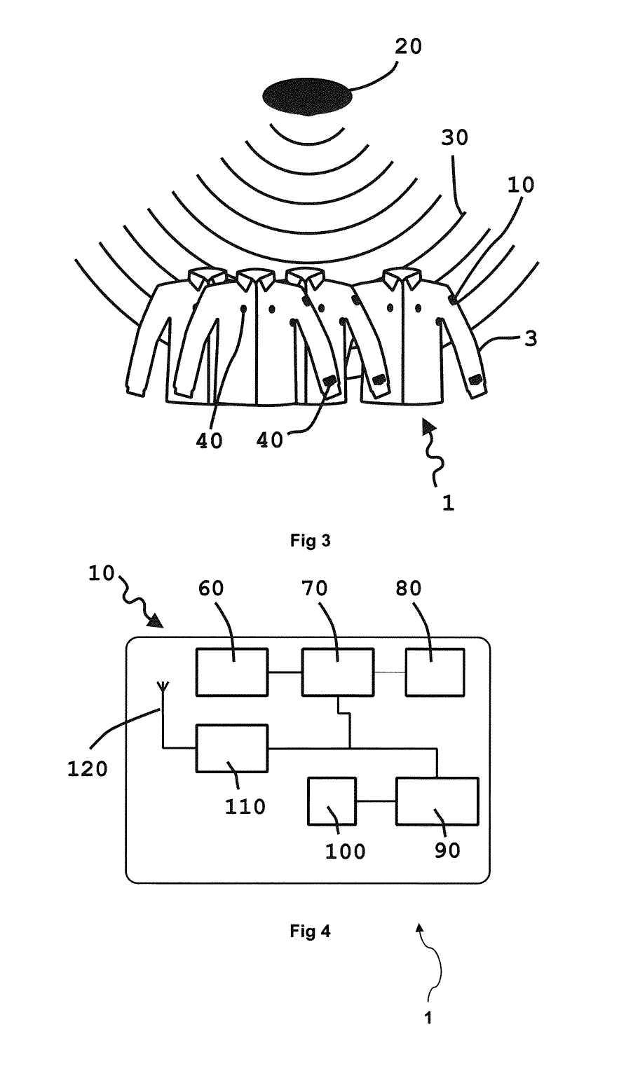

By reference to FIG. 1 (and FIG. 4) the first aspect of the invention comprises a system for sensing and logging physiological and/or environmental conditions of a wearer of an active uniform, the invention comprising a system of an item of active uniform 1 and a base station 2.

Active uniform 1 is comprised of uniform 3 for wearing by the user. It has been depicted as a shirt in the case of FIG. 1 however it could equally be a pair of trousers, a helmet, a shoe, a pair of sunglasses, gloves, etc. Uniform 3 is adapted to have mounted on or in it, electronic sensors 40 for sensing an aspect of the wearer's environment and/or of the wearer's physiology. Active uniform 3 also comprises an active tag 10. The active tag contains various components that allow it to communicate with base station 2 and send to it all of the sensor data stored in the active tag 10 which has been generated through the use of the electronic sensor or sensors 40.

Specifically, the active tag 10 comprises:
a sensor interface 80 for interfacing one or more electronic sensors to the active tag,
a microcontroller 70 that is programmed to (i) process the signals received over the sensor interface from the at least one electronic sensor into sensor data, (ii) send and receive sensor data, and identification data which identifies the wearer, to a base station transceiver, and (iii) send/receive and/or process instructions sent or received from the base station, including where necessary, the control of other components of the active tag,
a data store 60 including non-volatile flash memory for recording and storing sensor data and (optionally) identification data, radio frequency interface 110 adapted to convert electrical signals output by the microcontroller 70 into radio signals adapted to be received by the base station transceiver 320 and to convert radio signals received from the base station transceiver 320, into electrical signals that are adapted to be received by the microcontroller 70,
radio frequency interface 110 which may employ conventional RFID technology, whereby the information to be transmitted to the base station is encoded via the modulation of radio waves reflected from the tag, or an active transmitting technology, such as Bluetooth. The radio frequency interface may also include an electrical matching network to optimise the transfer of power to and from the antenna,
at least one tag antenna 120 for communicating via radio signals with the base station 2; and
power supply, in this case a battery 100 and power management unit 90.

The sensors 40 and active tag 10 are adapted to be retained on or in the item of uniform 3 such that the active uniform 1 is able to be washed without removing the electronic sensors 40 or the active tag 10.

The base station 2 comprises:
a base station antenna 20,
a base station transceiver 320; and
a data processing apparatus 340 connected to the base station transceiver, which is adapted to receive and optionally store data transmitted by the active tag of the active uniform, including at least, sensor and identification data.

The system of the active uniform 1 and base station 2 is used to collect data from wearers of the at least one item of active uniform which allows an assessment of the environmental or physiological risk to the wearer where said assessment is subsequently used to enhance the survivability of the wearer.

The transceiver 320 is connected to the data processing apparatus 340 via connection 330 which may be local (wired or wireless), for instance, a USB, UWB, Bluetooth, or other long distance networks such as radio networks, LANs, WANs, satellite networks, military radio services or even the Internet.

The active uniform 1 will gather sensor data in the field which is logged and uploaded into the base station's 2 data processing apparatus 340 when the wearer returns to base. Alternatively antennas 20 and transceivers 320 may be placed in vehicles including boats and ships or otherwise placed in the field, coupled with long range communication capabilities (or alternatively with mobile data processing apparatus 340 such as a laptop or tablet computer or a device such as a smartphone which is capable of communicating directly with the active tag 10 via Bluetooth or other wireless protocol utilised by the active tag 10).

For instance it may be envisaged that in the case of a chemical spill, emergency response personnel wearing active uniform may place mobile transceivers 320 (coupled with long distance communications capabilities or coupled with a laptop computer locally) at the entrance to a building in which the chemical spill has occurred so that all wearers of the active uniform 1 are accounted for and not left in the building where one or more may have lost consciousness or become trapped.

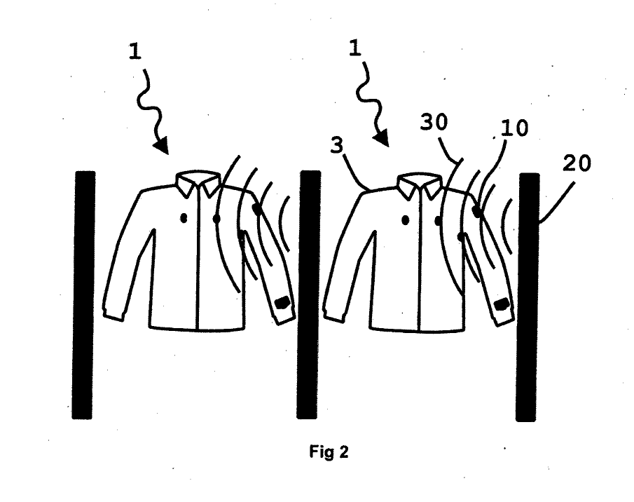

Referring to FIG. 2, active uniforms 1 incorporating active tags 10 are shown in close proximity to base station antennas 20 and the radio field 30 generated by the base station antenna 20. This is an example of a short range base station antenna 20. It would be suitable for narrow and confined spaces such as doorways and other passageways including hatches.

In FIG. 3, a longer range base station antenna 20 is depicted in which the wearers of active uniforms 1 comprising active tags 10 and sensors 40, are enveloped by the radio field 30 generated by the base station antenna 20.

Alternatively mobile base station antennas 20 and base station transceivers 320 may be placed in vehicles including boats and ships or otherwise placed in the field, coupled with long range communication capabilities. For instance it may be envisaged that in the case of field action involving military personnel wearing active uniforms a mobile reader may be placed at the camp entrance so that all personnel are monitored. Alternatively in the case of ships, readers could be placed at the perimeter of the deck for use in rapidly identifying a man-overboard situation.

Multiple base stations may also be used where the data processing apparatus 340 from each base station 2 is adapted to integrate the collected data (sensor data and identification data) into a single data store, or alternatively, there may be multiple data stores of collected data in each data processing apparatus 340 which are in communication with a further data processing apparatus (not shown) for subsequently combining the data and storing the combined data for analysis.

Active tags 10 need power to run the electronic sensors 40 and other electrical components. Power can be delivered to the active tag 10 in one of a number of ways. Power can be provided by a battery 100, and power management unit 90 as shown in FIGS. 1 and 4. Alternatively it is also possible to power the active tag 10 and associated electronic sensors 40, by way of power harvested from the wearers use of the active uniform 1 through microelectrogeneration. Further, it is possible for such approaches to be combined such that the active tag 10 possesses a battery 100 but this is a rechargeable battery that is charged by microelectrogeneration, or the active tag 10 has a second, rechargeable battery or electricity store such as in a capacitor, in addition to battery 100, for receiving power generated through microelectrogeneration.

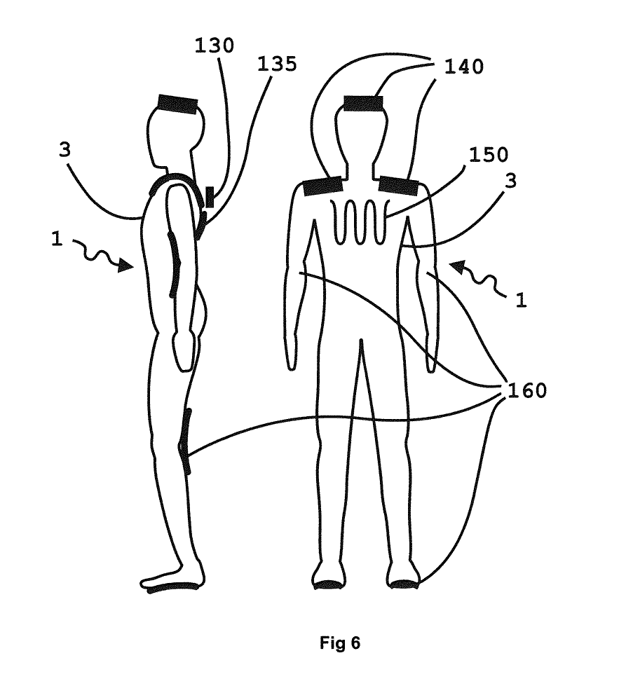

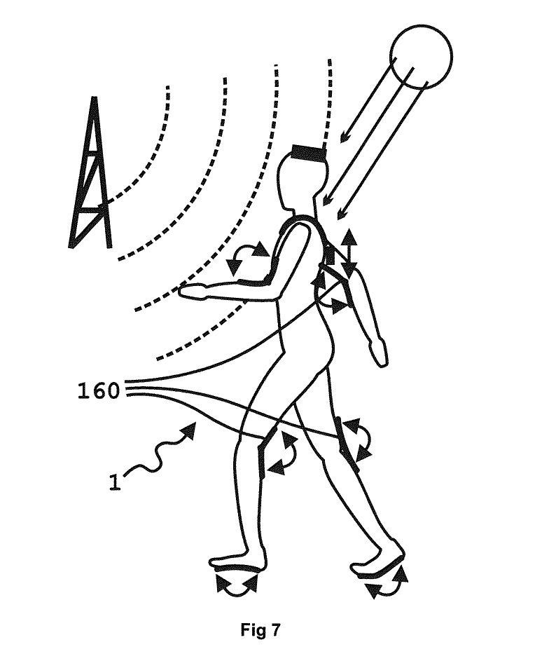

FIGS. 6 and 7 depict examples of microelectrogeneration apparatus that operate to power the active tag 10. For instance, solar energy harvesters 140, radio field energy harvesters 150, heat differential harvester 135, motion energy harvesters 130 and 160 are indicated that can be utilised to replace or supplement the battery 100. Energy-Harvesting controllers such as Maxim's MAXI 7710, Linear Technology's LTC3588-1—Piezoelectric Energy Harvesting Power Supply and Piezo Systems Inc' piezoceramic transducers can be supplemented with solar panels and RF collecting antennas used in this implementation. Other sources of electrical energy such as rechargeable fuel cells may also be utilised, some of which may be engineered to run on glucose derived from the wearer's bodily fluids leading to embodiments of the invention that may be miniaturised and implanted in the body of a soldier, for instance. Other alternatives include inductive charging of an internal power supply such as a battery 100 wherein the tags may be recharged during the laundering process using an inductive charger.

The active tag 10 of the active uniform 1 may employ conventional RFID tag technology and communication protocols. However, whilst it may employ similar technology to power a transmitter and transmit information, it is important that the active tag 10 only responds or transmits information in response to base station transceivers 320 that have been authenticated or otherwise known to not be compromised. In a military setting, the active tags 10 must not allow a soldier to be identified more readily than otherwise would be the case. In effect it requires the active tag 10 to be radio silent and only respond to trusted base stations 2. In the RFID technology, communications are covered by the ISO/IEC18000-6 standard. This standard requires that tags have unique IDs and provide how that is implemented. In a secure, military application, the lowest level command descriptions are changed to be different from valid commercial use. This can be implemented by reallocating command numbers between those that are commercially used and/or by changing the CRC checksum calculation. These require a change to the lowest level of the RFID chip implementation and reader protocol.

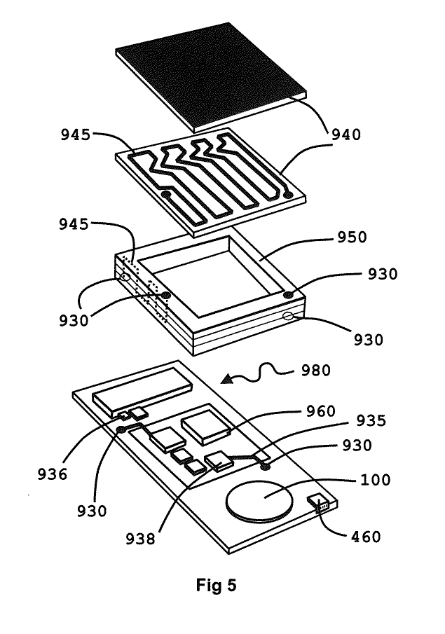

The active tag 10 is tamper resistant. In particular it is adapted to erase the contents of the memory of the device upon opening as well as any other component that may identify the wearer or store any other operational information. In FIG. 5, the tamper protection elements consists of two additional circuit boards over the main electronic circuit board 980, namely multilayer cover board 940 and wall board 950, that form an enclosure around the electronics 960 being protected. Cover board 940 and wall board 950 have conductive tracks 945 formed in them. Additional planes and vias of copper are implemented to hide the position of the actual conductive tracks 945. Electrical connections 930 are provided between the PCBs and are internal and hidden from view. The long and hidden conductive track 945, and the shorter conductive track 935 formed on main electronic circuit board 980 form a circuit and are adapted to indicate tampering as any attempt to pierce through it will break the circuit and will be detected by microcontroller 70. The detection method is as follows; one side of the track 935 is connected to the battery 100 ground (GND) and the other side is connected to an input to the microcontroller 70. These components are connected electrically at electrical connections 930 by way of hidden track 945. The microcontroller 70 will pull up and read track 935. If the value is zero, this means the protection is still connected, otherwise something or someone is trying to access the hardware. The microcontroller 70 needs to read the protection value repeatedly which can be done using interrupts or by polling. Using the microcontroller's 70 IO pin (not shown) for pulling up the protection is chosen to reduce the power consumption. In this case the microcontroller 70 can periodically output a high level on the output for a short amount of time.

In the case an attempt to access the contents of the active tag 10 is detected, the microcontroller 70 will delete its code and any stored data to protect it from being revealed. It does this by supplying an instruction to delete and overwrite data in the flash memory 60 and firmware and memory of the microcontroller 70. To prevent this function from being stopped by way of the removal of the battery that supplies power to the microcontroller 70 and flash memory 60, there is also supplied, a capacitor 938 that stores sufficient power to carry out the deletion of the data and operational information.

In addition to the anti-tamper and anti-reverse engineering features described above, the active tag 10 is fully encapsulated in a non-conductive, radio transparent material that forms a water-tight, chemical resistant seal around the electronics, typically an epoxy potting compound such as MG Chemicals 832B which is non-porous, water and chemical resistant, extremely impact resistant (contains a form of nylon), coloured black to prevent visual inspection, affords high security as once cured it is extremely difficult to remove, is non-conductive, is an electrical insulator and is of low toxicity. Masking materials such as solder mask or adhesive tape may also be applied on or around certain components to minimise the adhesion of encapsulating material and so assist with their removal for servicing after encapsulation.

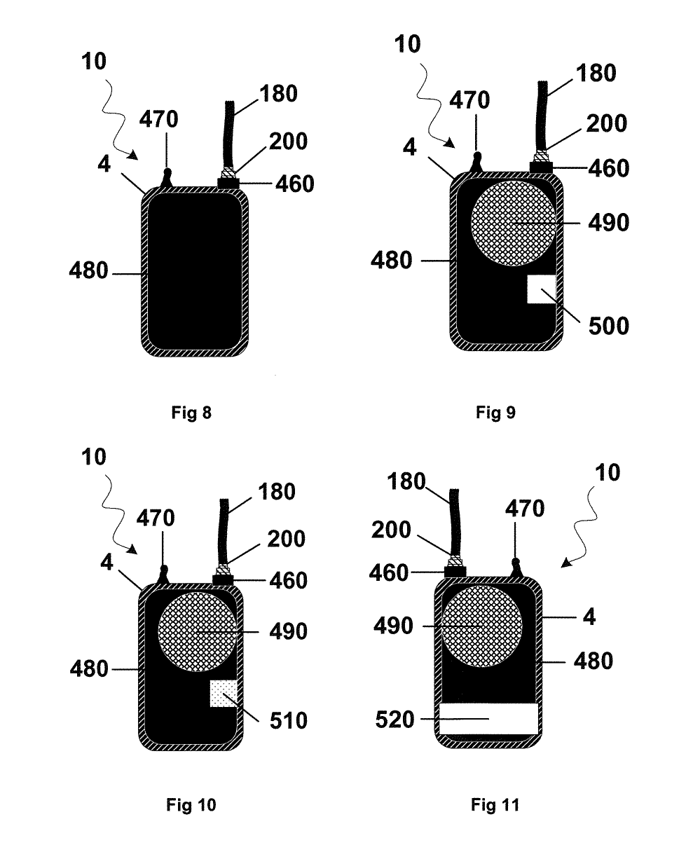

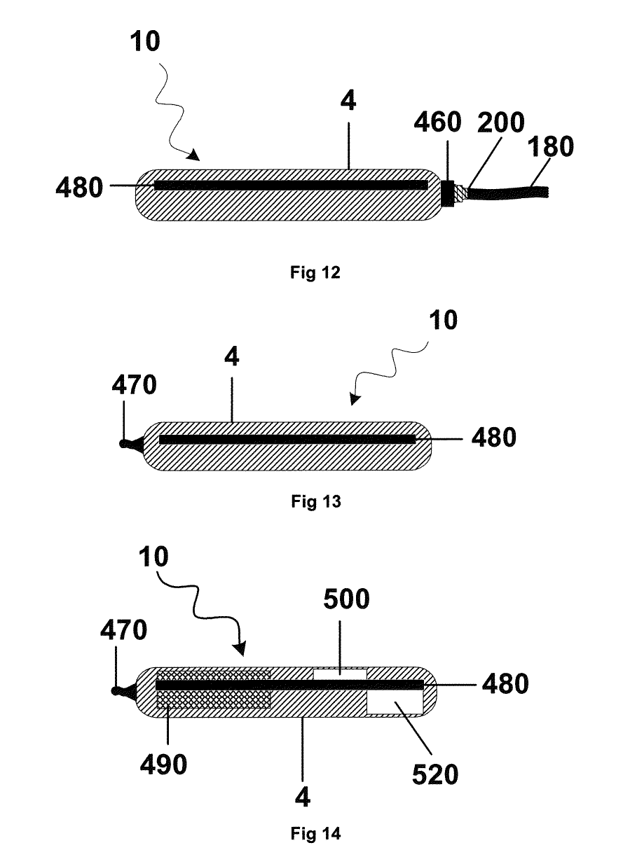

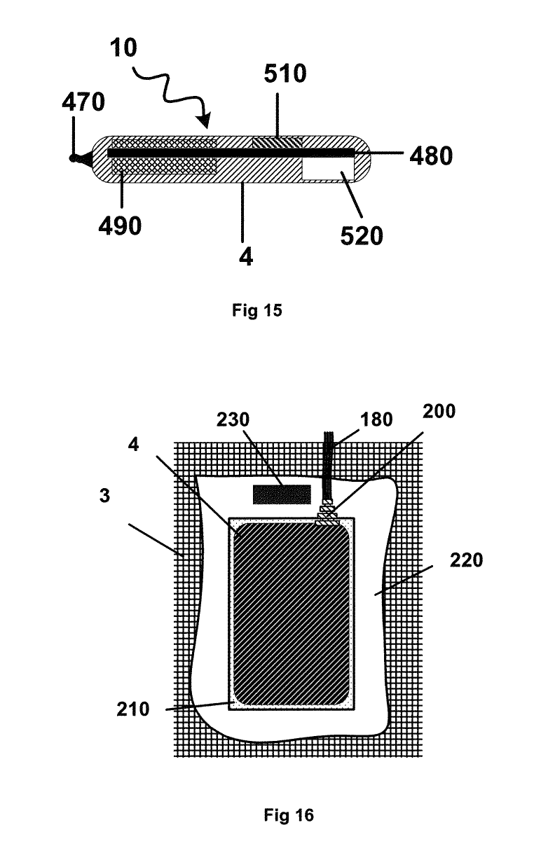

The tag antenna 120 and the transceiver of the radio frequency interface 110 of the active tag 10 requires matching for optimization. This matching is achieved by small components near the antenna. Materials surrounding the matching components or the tag antenna will impact how well the tag antenna is matched. For lower manufacturing volumes where tags will be tuned to one of a number of communication frequencies and possibly small quantities, the inclusion of air gaps around the antenna and matching components dramatically reduce the complexity of this optimization process. This also helps to keep inventory costs down. For higher manufacturing volumes where tags are matched for a particular frequency and the matching component values are known, the air gaps may be excluded. This simplifies the encapsulation process. Examples of the foregoing are provided as depicted in FIGS. 8 through 15 (where it should be noted that the air gaps 500 and 520, foam 510, masking material 490, antenna 120, network matching components 936 and battery 100 all sit outside of the tamper proof monitoring zone bounded by main electronic circuit board 980, multilayer cover board 940 and wall board 950):
Encapsulating an air gap 500 above the tag antenna 120 matching network components 936 (depicted in FIG. 5) which are part of the radio frequency interface (enclosed in epoxy to maintain a seal to the environment) (FIGS. 9, 14);
Encapsulating a portion of foam 510 around the tag antenna matching network components 936 (enclosed in epoxy to maintain a seal to the environment) (FIGS. 10, 15);
Encapsulating the tag without an air gap or foam around the tag antenna matching network components 936 (FIGS. 8, 12);
Encapsulating an air gap 520 under the antenna 120 to improve radio frequency transmission efficiency (enclosed in epoxy to maintain a seal to the environment) (FIGS. 11, 14, 15);
Encapsulating a “masking” material 490 (an easily removable material such as printed circuit board solder mask) around the battery to allow battery replacement (or removal for legal disposal of the lithium ion battery) by a trained technician (FIGS. 9, 10, 11, 14, and 15)
Encapsulating the battery without a masking material so that it is difficult to remove and tamper with (FIGS. 8, 12)
Encapsulating the tag in such a way, with an internal (and hidden) airgap, as to provide a port 470 for accessing the programming pins of the microcontroller 70 (where the port/pins are only accessible to a technician, for testing and servicing the active tag. The hidden internal airgap can be accessed with a knife with knowledge of the location and resealing process (by cutting away the section of epoxy that covers the air gap at a hidden/unmarked location) (FIGS. 8, 9, 10, 11, 13, 14, 15).
Encapsulating any cable exiting the active tag with a strain relief 200 (in embodiments that call for external electronic sensors or external power supplies including the first and third embodiments);
Encapsulating any cable exiting the active tag with a connector 460 (in embodiments that call for external electronic sensors or external power supplies that can be detached from the active tag 10 including the first and third embodiments).

Cable connector 460 and ends of the conducting cables 180 are designed such that they are only removable by authorised service representatives. This can be achieved by creating or using fasteners or fastening techniques that require specialised tools which are not generally or commercially available.

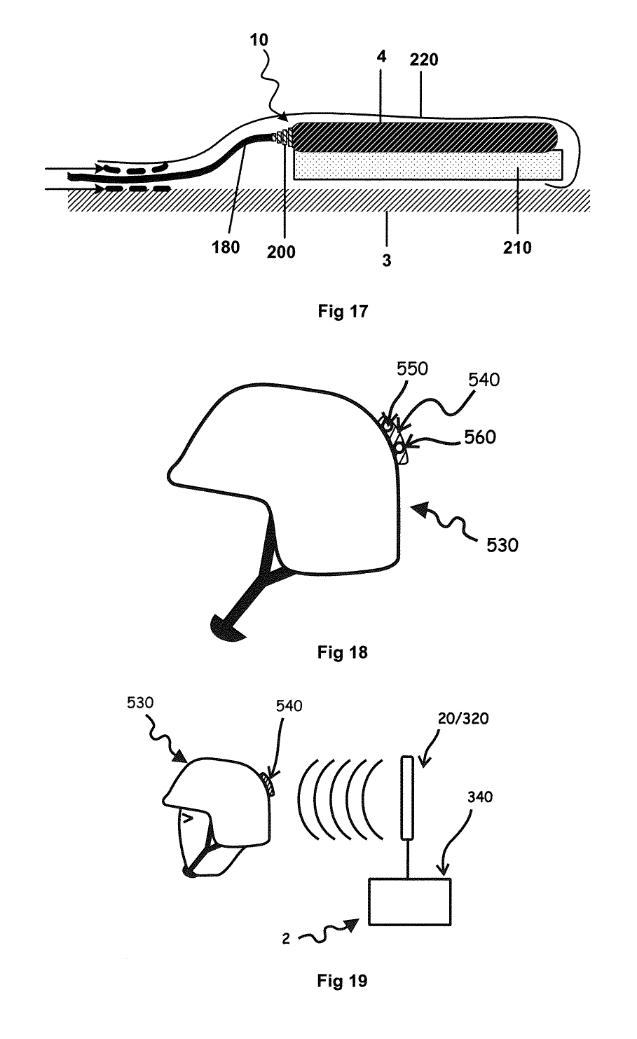

Degradation of the radio communication via the antenna 120 can also be contributed by close proximity to the wearer's body. For this reason, in FIG. 16 and FIG. 17, the active tag 10 is depicted in use located on top of a spacer 210 that consists of many air cavities (such as a light weight polymeric foam). The active tag 10 and the spacer 210 may be placed in a pocket 220 sewn on the outer surface of the uniform 1 and can be secured in the pocket via a loop and hook fastener 230, or alternatively, permanently sewn into place by sewing the pocket shut once the active tag 10 has been inserted.

Electronic sensors 40 may be of two general types, internal to the active tag 10 (as in the case of the second embodiment of the invention), or external to the active tag 10 (as in the case of the first embodiment of the invention), or a combination of both internal and external (as in the case of the second embodiment of the invention).

Generally, the type of sensor will dictate whether it would be suitable for inclusion within the body of the active tag's 10 container. For instance, an accelerometer may be suited for internal use within the active tag 10 whereas a chemical or biological agent assay would need to be necessarily exposed to the environment in order to perform their desired function.

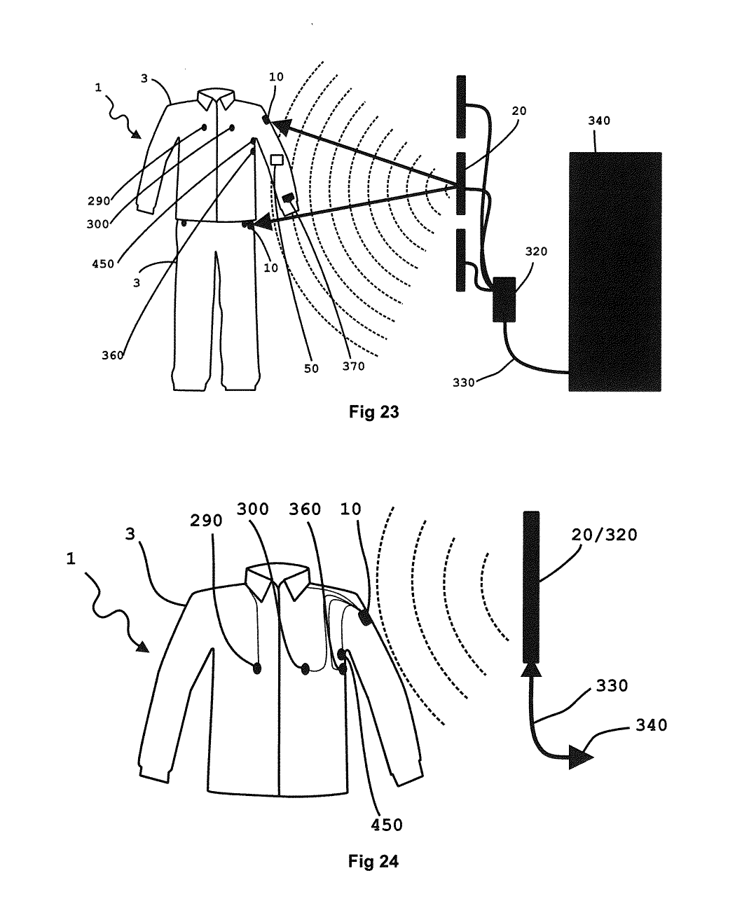

Shown in FIG. 23 are two pieces of active uniform 1 in accordance with the first aspect of the invention (pants and shirt). With respect to the shirt active uniform 1 there is embedded active tag 10 and various external sensors including ECG sensors 290, 300, and 360, temperature sensors 450, chemical sensors 50, and an active assay biological sensor 370. Immunoassays are a standard approach to detecting a chemical or other material. An assay is a chemical that will take part in reacting with the material to be detected, in such a way that you can measure the reaction chemically, optically, magnetically or even acoustically.

Other sensors 40 may be incorporated into active uniforms 1 of the present invention including:
GPS and/or accelerometers for determining the location and/or motion of the wearer
sensors for determining the respiration of the wearer
sensors for determining the blood pressure of the wearer
sensors for determining the blood glucose levels of the wearer
sensors for determining the impacts or forces imparted on the wearer
sensors for determining radiation dosage to the wearer
sensors for determining the ambient temperature, humidity or barometric pressure
sensors for determining the number of wash cycles of the garment
sensors for determining the degree of fading of the garment.
sensors for determining the brain activity of the wearer (EEG)

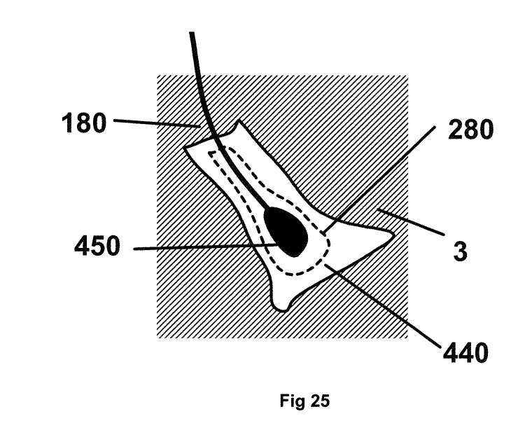

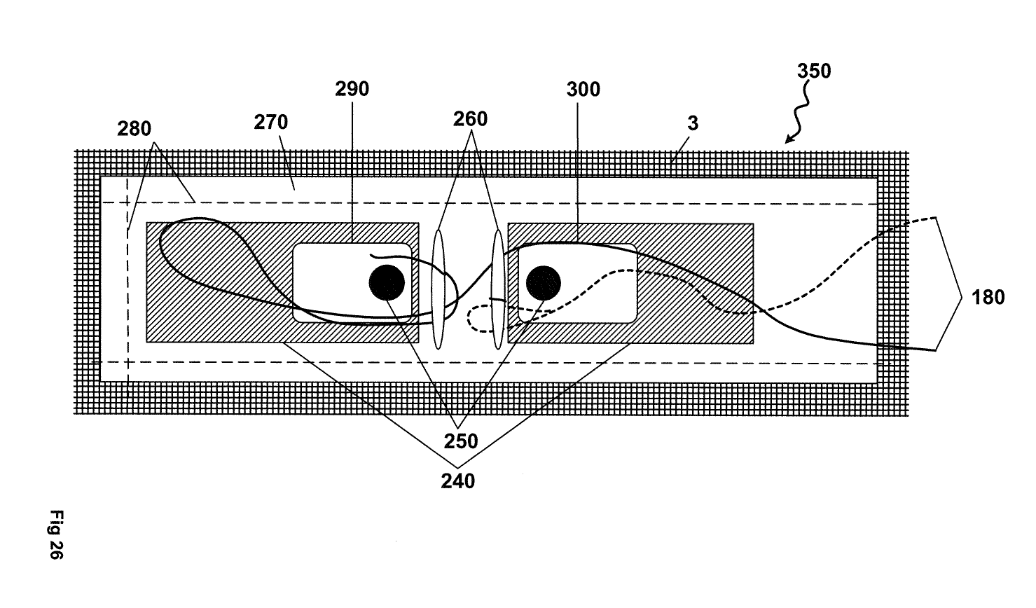

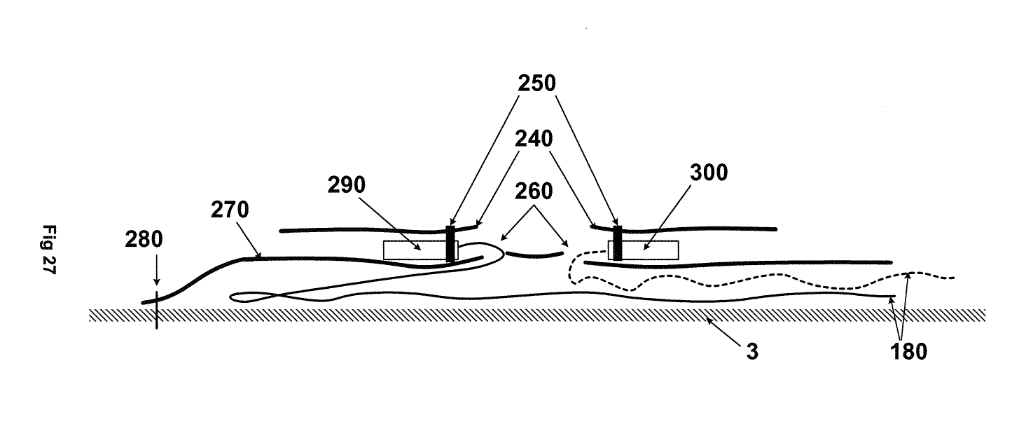

Sensors may be conventional in that they are discrete, encapsulated components that can be attached to, sewn into or embedded into the garment (see tunnel 270 in FIGS. 26, 27) or pockets provided in the garment (see pocket 440 in FIG. 25). Alternatively they may be comprised of or contain smart fabrics which typically have integrated circuits and/or other electronic or optically responsive elements that are woven into the fabric and are capable of responding to physical stimuli and provide electrical and/or optical signals in response. Such smart fabrics include the material produced by Textronics, Inc (now Adidas Wearable Sports Electronics). This material is a breathable weave 75% nylon 21% spandex (elastane) with embroidered patches of an electrically conductive thread.

External sensors are coupled to the active tag 10 via conducting cables 180 (FIGS. 25 to 30 and FIG. 36). The conducting cables 180 may exit encapsulated active tag 10 via a cable strain relief 200 as shown in FIG. 17.

The cables may also exit the encapsulated active tag 10 via a connector 460 and cable strain relief 200 as in FIGS. 8 to 12 if it desired that the cables be able to be disconnected and reconnected to the active tag 10. In such cases, conducting cables 180, cable connector 460 and cable strain relief's 200 are waterproof, medical grade components such as those available from PlasticsOne, a medical component manufacturer, or those utilised in medial devices such as pacemakers and other implantable electronics.

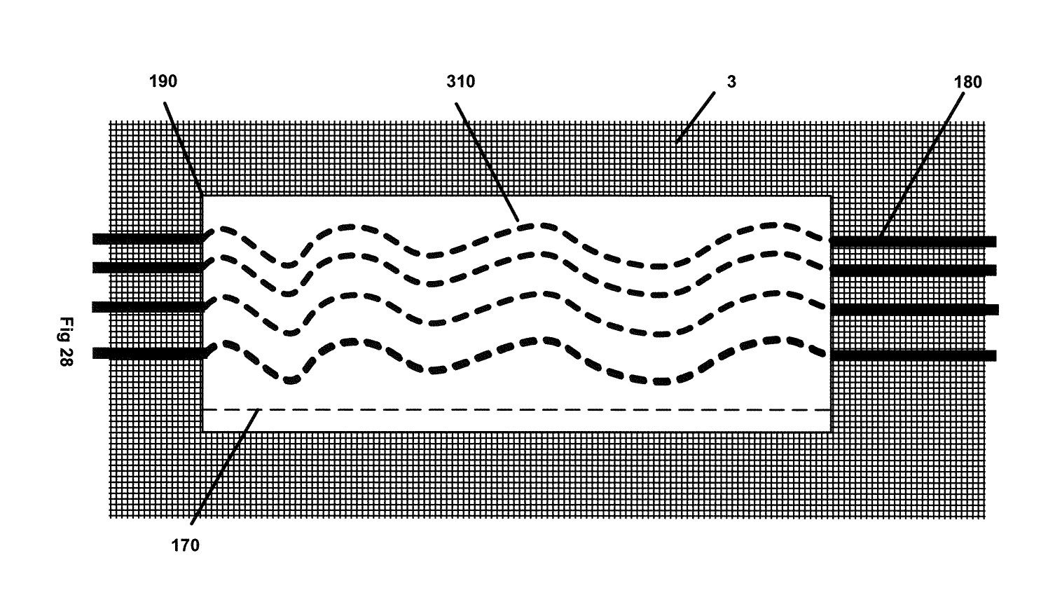

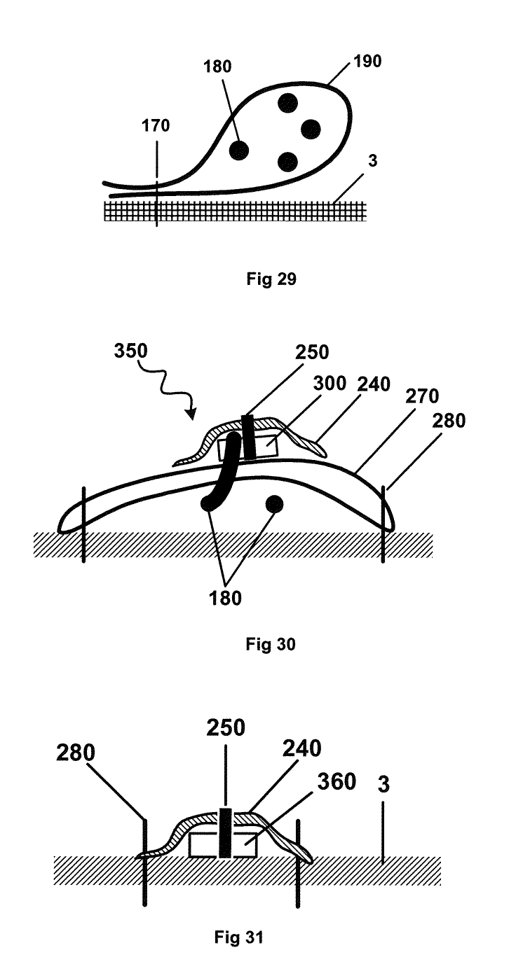

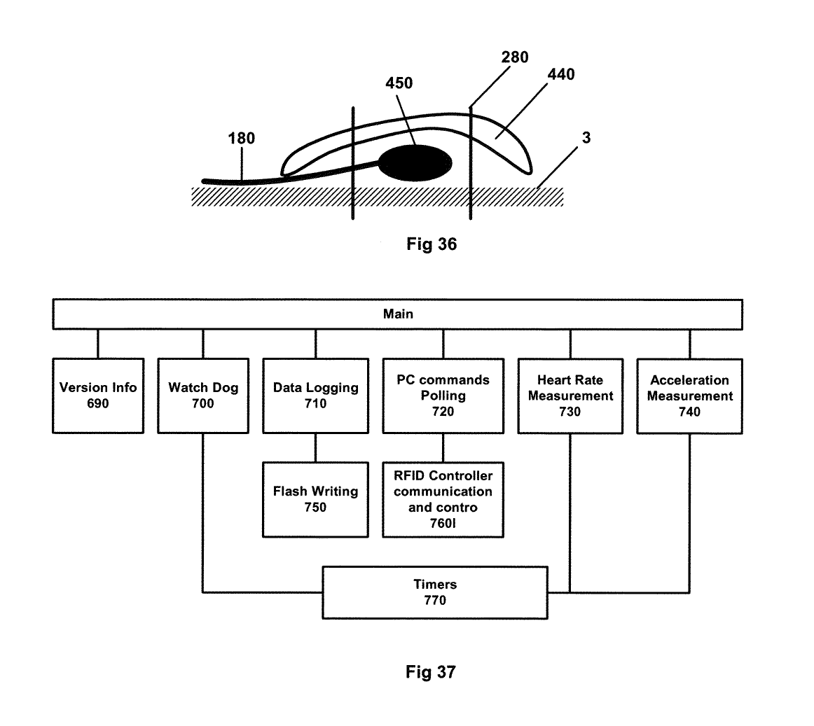

As a result of the inclusion of waterproof active tags 10, sensors 40, cables 180 cable relief strain 200 and cable connector 460, the electrical components of the active uniform 1 are themselves as a system, waterproof and as a result are able to be sewed permanently into fabric tunnels and pockets such that the active uniforms 1 can be washed in a commercial washing machine and dried in a commercial dryer without first removing the electrical components. FIGS. 25-36 all depict the various ways in which external sensors and active tags are accommodated in the uniform 3. Cables 180 are shown threaded through a network of fabric tunnels 190 and 270 to ensure the cables do not catch on other parts of the uniform 1. The cables are arranged in a flexible and stretchable configuration to allow wide bends 310 in the cables as shown in FIG. 28. The tunnels 190 and 270 may be sewn on one side 170, FIGS. 28 and 29, or they may be sewn on more than one side 280, FIGS. 26 and 27. For example, the temperature sensor 58 in FIGS. 25 and 36, is sewn with stitches 280 into a pocket 440 on the uniform 3. It is placed in the underarm area of the uniform 3 shown in FIG. 23 to provide a close approximation of the wearer's body temperature.

Depicted in FIGS. 26 to 36 are an array of ECG sensors which utilise a smart fabric, and in particular, the Textronics material detailed above. In these figures the ECG sensors 290 and 300 are securely connected via eyelets 250 to fabric electrodes 240 made from Textronix material, for the chest. The fabric electrodes 240 are attached to a cable tunnel 270 that is sewn horizontally across the chest of the uniform 1 on the inside surface. The cables 180 to the ECG sensors 290 and 300 are run through the cable tunnel 270 in a flexible and stretchable configuration as depicted in FIG. 28. The cables 180 exit the tunnel 190 via two slits 260 in the tunnel near the ECG sensors 290 and 300. The third ECG sensor 360 is securely connected to the fabric electrode 240 via a connector formed like an eyelet 250 in FIG. 31. The fabric electrode 240 is sewn directly onto the inside of the active uniform 1 creating a pocket in which the third ECG sensor 360 sits.

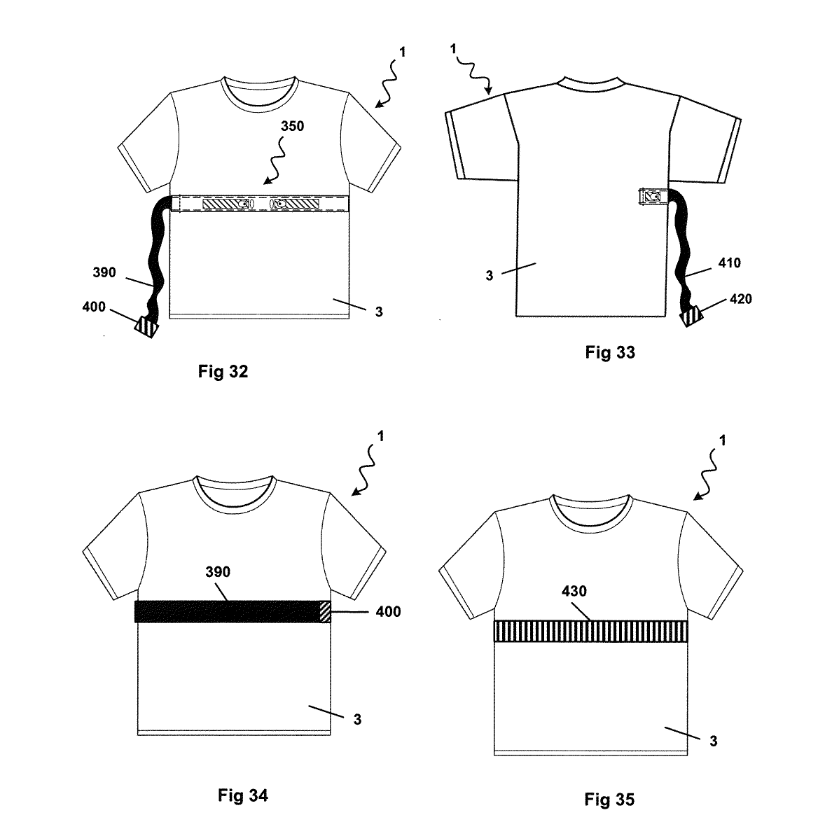

The ECG sensor assembly 350 comprising of the ECG electronic sensors 290, 300, 360 and fabric electrodes 240 are held firmly against the wearer's body to reduce the effect of the wearer's movement on the ECG readings. This may be achieved via flexible elastic straps, namely front elastic strap 390 and back elastic strap 410 attached to the active uniform 1 as shown in FIGS. 32 to 34. The front elastic strap 390 and back elastic strap 410 may be connected together via fasteners 400 and 420. These fasteners 400 and 420 may be of a hook and loop configuration. Alternatively, the strap may also be a band of elastic material 430 integrated into the active uniform 1 shown in FIG. 35, or a belt worn over it. There are benefits to having the straps integrated within the garment fabric (as in FIG. 35), however; there are disadvantages such as the garment will always be tight and it will be more difficult to put on and take off. The implementation in FIGS. 32-34 is easier to use from the user's perspective as the garment can be easily put on, taken off and worn without discomfort and the tightness of the strap can be more readily adjusted to the individual wearer.

In use the ECG voltage between the two chest electrodes 290 and 300 is amplified then sampled by the tag 10. The third electrode 360 contacting the skin in the underarm area is connected so as to provide an earth reference voltage for the amplifier, in order to reduce common mode mains interference.

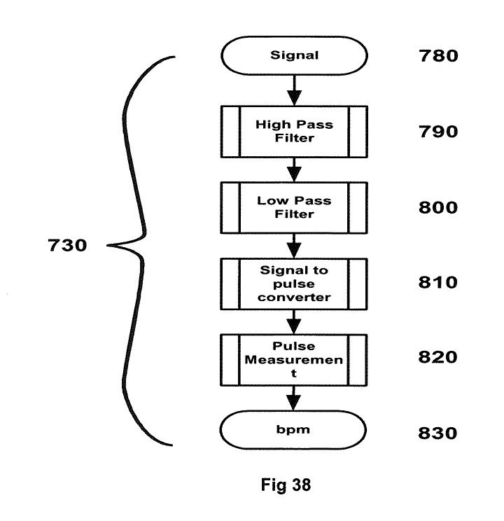

The following description is of the aspects of the firmware that adapt the microcontroller 70 to be able to read ECG signals 780 in FIG. 38 and convert them into a heart rate 830 for logging in the data store 60. As shown in FIG. 37, the firmware of the microcontroller has a variety of modules including timers for control of sensor sampling intervals 770, communication and ID control module 760, module for storing logged data to FLASH 750, module for determining shock or movement 740, module for determining pulse rate 730, module for formatting sensor data and time stamping of data 710, module for firmware failsafe 700 and version control functions 690.

In use, timers 770 sample the ECG signal 780 coming through to the microcontroller's 70, I/O pins. This signal might already have been processed or filtered by the hardware in the ECG sensors 290, 300 and 360. In any case, the firmware of microcontroller 70 uses a high pass filter 790, indicated in FIG. 38, and a low pass filter 800 to make the ECG signal more suitable for measurement. These are implemented in the microcontroller firmware to limit the ECG signal frequency range to improve the signal-to-noise ratio and attenuate mains-induced interference, while still providing an appropriate bandwidth to reliably detect the QRS feature in the ECG signal. The ECG signal 780 is sampled by the microcontroller at approximately 115 samples per second as this permits good rejection of mains interference near frequencies of 50 Hz, 60 Hz and 400 Hz, which cover all commonly used ground and aircraft power frequencies. This is achieved by tailoring the low-pass filter 800 to have an attenuation of greater than 60 dB at 50 Hz, which is within its normal input signal frequency range as determined by the sampling frequency. In addition, any interfering signals at 60 Hz and 400 Hz are, when sampled, aliased to frequencies where the filter attenuation is also greater than 60 dB.

As indicated in FIG. 38, the firmware may convert the ECG signal to a pulse 810, in FIG. 38, for measurement purposes. The firmware can sample the ECG pulses 810 for a certain amount of time and use the median of the pulse measurements as the calculated ECG period. Firmware algorithms 810 and 820 implemented in microcontroller 70 estimate the wearer's heart rate 830 using the period between successive occurrences of the QRS feature in the ECG signal. Start and end times of the QRS feature in each heartbeat cycle are estimated using an adaptive threshold detection algorithm which incorporates information from the peak amplitude of the QRS feature in previous heartbeat cycles. The measured time intervals between successive QRS start times and successive QRS end times over a period of typically 5 to 10 seconds are used to calculate a statistical estimate of the average heart rate over this period, as well as a figure-of-merit representing the reliability of the heart rate estimate. The figure-of-merit will depend on the quality of the ECG signal as determined by electrode contact and body movement effects, residual mains frequency interference and other external interfering signals.

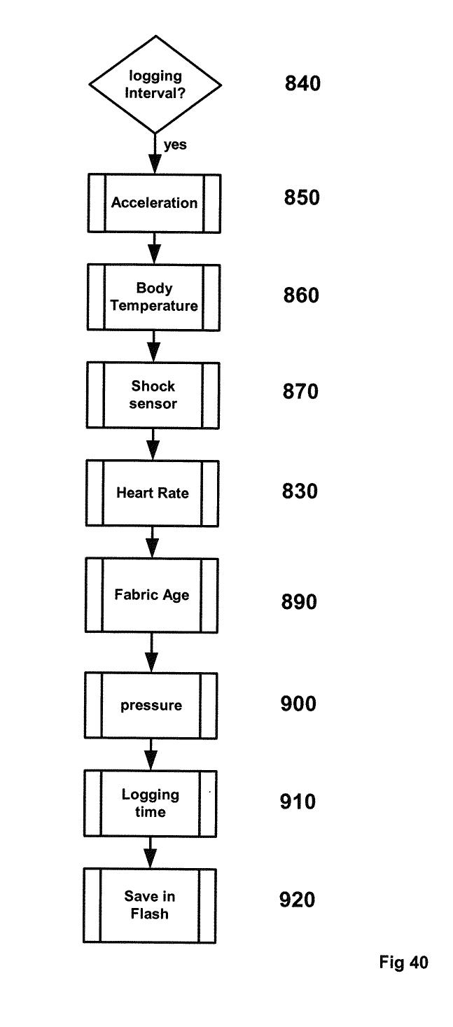

As shown in FIG. 40, the firmware may read the calculated values for acceleration 850, body temperature 860, shock events 870, and heart rate 830 to construct a data sample for logging. The firmware adds a time stamp 910 to each data sample to be saved in the log or may save the start time and use logging time interval to work out sampling times. The firmware may write 920 the logged data in the internal or external flash memory 60.

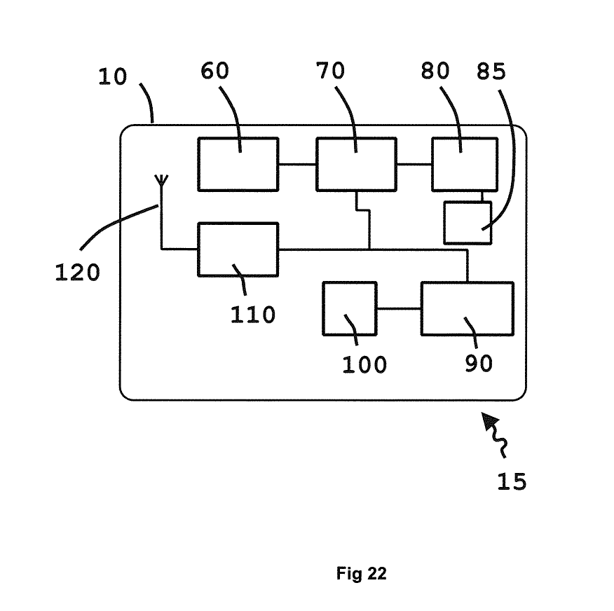

The following description is of the second embodiment of the invention depicted in FIGS. 22 and 24. The second embodiment of the invention involves active tags 15 which incorporate electronic sensors internally in the active tag 15 in addition to the external sensors described by reference to the first embodiment and depicted in FIGS. 1 and 23.

Referring to FIG. 22, in the second embodiment of the invention, the active tag 15 has an internal accelerometer sensor 85, in this case a three axis MEMS type electronic accelerometer contained within the active tag 15, which is used to gather information about the relative movements of the wearer of the active uniform 3. FIG. 24 depicts active tag 15 in combination with ECG sensors 290, 300 and 360, and temperature sensor 450, all of which are mounted or sewn into uniform 3 which in the present case is a shirt or top.

The following advantage of providing an accelerometer, ECG sensors and temperature sensors, in association with an active tag, as provided in the second embodiment of the invention, is described by reference to the second embodiment of the invention, however it is broadly applicable to any system in which there are a plurality of sensor types, including the first and third embodiments of the invention.

Heat stress becomes an issue when the body temperature rises above 35-38 degrees C. and there is no way for the subject to cool down due to the environment and the effort being expended. There is no electronic sensor that can provide, on its own, a measure of the heat stress experienced by the wearer of an active uniform. However monitoring heart rate, physical activity (as indicated by X Y Z accelerometers) and rising temperature of the subject are used to determine the point above which heat stress is likely. Thus the microcontroller 70 (or the data processing apparatus of the base station) is able to log manipulated and/or converted sensor data from the plurality of sensors, to determine a measure of the wearers physiology or environment that is not otherwise directly measurable by any one sensor alone.

The multiple sources of sensor data also enable the microcontroller 70 (by way of algorithms in the microcontroller's 70 firmware) to only log what is more likely to be accurate data. For instance, during periods of high movement it would be less efficient to measure temperature and heart rates as the reliability of the readings could be affected by the wearers own movements. The active tag 10 microcontroller 70 may as a result incorporate algorithms combining multiple sensors to improve the accuracy and robustness of the sensor readings. In monitoring the heart rate, accelerometer sensors may be used to indicate when the best quality signal from the heart rate sensors is available. As skin and muscle movement may result in a less reliable heart rate signal, accelerometer readings can be used to determine when this is not occurring. This can be expanded to other combinations of sensors and appropriate algorithms.

As can be seen from the above two examples, the provision of a plurality of different sensors can aid in the (i) the reading of an aspect of either the environment or the wearers physiology in the absence of a specific sensor for measuring that what is sought to be measured, and (ii) for taking more accurate measurements. However in order for the active tag to perform these more sophisticated calculations and performance of steps in algorithms, the microcontroller 70 of the active tag 1, needs to be programmed with specific firmware, using conventional techniques.

A third embodiment of the invention as depicted in FIGS. 18-21. In this embodiment the active tag 540 houses all electrical components including all sensors. It is particularly adapted to be mounted on or in a helmet for use by the military and hazardous environment civil and industrial workers. The embodiment would also be suitable for placement on other parts of the wearer's body such as in a garment or on footwear, however it is has been described by way of reference to a helmet. This is not intended on being a limitation, however.

One particularly useful application of this embodiment is the use of an active tag for the monitoring of the wearer's exposure to shocks and blasts. Such information is useful to identify personnel that have experienced significant shock which may then require the person to be pulled from active duty, or indeed, treated. The data may also be useful in the future for developing models for investigating and estimating brain trauma injuries and assessing medical claims made by personnel for conditions associated with blast injuries.

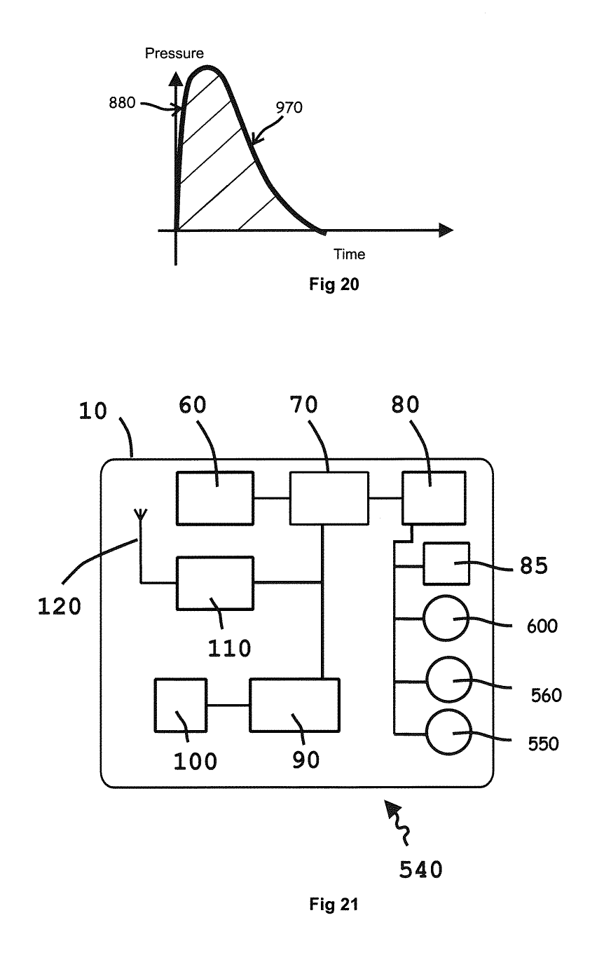

An active helmet 530 (essentially an item of active uniform 1) is shown in FIG. 18 and FIG. 19 with an active tag 540 mounted on its rear. As in the case of the previous embodiments, the active helmet 530 is part of a system including a base station 2. The blast wave from an explosion is typically characterised by a very rapid rise 880 in atmospheric pressure, followed by a slower decay 970 as shown in FIG. 20. The active tag 540 incorporates one or more blast/pressure sensors 550 and 600, mechanical shock sensor 560 all of which respond to rapid changes in atmospheric pressure and/or mechanical shocks to the wearer's head, including those that might be caused by an explosive blast. Multiple blast/pressure sensors are provided (550 and 600) with difference sensitivities to account for the large range of dynamic range possible as a result of a blast. The active tag 540 also comprises accelerometer 85 and other components as described by reference to FIG. 1 or 4.

The pressure/blast detectors 550 and 600 may be an existing pressure transducer, such as produced by PCB Piezotronics, Knowles or Kulite using MEMS or piezo transducer technology; possibly with a mechanical attenuator. This may be implemented as a transducer attached to a metal plate, or behind an orifice plate within a chamber. How big and thick that metal plate is determines how big a blast can be measured. Multiple plates can be provided for each of the blast pressure sensors 550 and 600 that respond to different ranges of pressure/shock. Preferably piezoresistive transducers are used as they have the low frequency response required.

It is envisaged that the smart helmet 530 will gather data in the field which will be logged and uploaded into the base station's 2 data processing apparatus 340 when the soldier returns within proximity to a base station 2 as shown in FIG. 19 (the alternative embodiments also apply which utilise long range communications between transceiver 320 and data processing apparatus 340 as applied to the earlier embodiments).

The data transferred from the active tag 540 to the data processing apparatus 340 (which in the present example is a personal (programmable) computer or PC), may optionally be encrypted and compressed such that the computer software on the PC 340 connected to the base station transceiver 320 that receives the data from the active tag 540, decodes and decompresses it before entry into its database of data which can be any database but preferably a SQL database. The decoded data might have time stamps or may contain start up time and logging time interval. So time for each data sample can be calculated and saved in the database in addition to other data samples.

The signals from the sensors 85, 550, 560, and 600 of FIG. 21 are sampled directly by the microcontroller 70, or additional signal conditioning electronics may be interposed between the sensors 85, 550, 560, 600 and the microcontroller 70. The signal conditioning electronics are typically sensor signal gain adjustment, offset filtering and sensor biasing or powering. The sampling rate should be sufficiently high (for example, several thousand samples per second) so as to enable the measurement of particular features of a significant overpressure or shock event which may be relevant to the assessment of biological effects, such as peak and/or integrated amplitude, duration and decay rate.

A first system configuration incorporates sensors and other components with a combined power consumption low enough to allow continuous sampling of one or more of the overpressure and/or shock sensors 550, 560 and 600. Upon detection of a sensor signal level which exceeds a predetermined threshold, all subsequent sensor data samples are continually saved in flash memory 60 for either a predetermined number of samples, or until the signal level drops below another predetermined threshold. Thus each significant overpressure event results in the generation of a time-sequential data sequence saved in flash memory 60. To minimise data storage requirements and the time required to retrieve the data from the active tag 540, only sensor data associated with individual significant overpressure or shock events need be maintained in flash memory 60 for later retrieval.

An alternative system configuration (not shown) involves the incorporation of an extra overpressure and/or shock sensor and accelerometer, where it and its associated electronics are of such a design such that their power consumption is extremely low and thus can operate continuously. These sensors need only have sufficient accuracy to simply detect a rapid change in pressure or acceleration. In addition, the microcontroller 70 can be placed into a very low power (“sleep”) mode so that its power consumption is minimal. Since the pressure wave FIG. 20 from a blast typically exhibits a very rapid initial rise 880, the signal from this first sensor can thus be used to “wake up” the microcontroller at the onset of a significant overpressure event. The microcontroller 70 in FIG. 21 can then sample the output of a second, more accurately calibrated (and higher power consumption) sensor and continue to save the data in flash memory 60 for either a predetermined number of samples, or until the signal level drops below another predetermined threshold. The low power sensor in this case could incorporate a very low cost uncalibrated piezoelectric element with a sufficiently fast response time.

These possible configurations are not intended to be exhaustive, and it is apparent that other similar sensor types, sensor combinations utilising multiple sensors and detection schemes to minimise power consumption and data storage requirements whilst maintaining accuracy could be easily envisaged. For example, algorithms implemented in the microcontroller could combine signals from multiple sensors, provide filtering or data compression functions, or ignore data from spurious events.

In the embodiments of the base station 2 described above, the data processing apparatus 340 is described variously as either a programmable computer, or a standalone data storage and communications device which is programmed by way of firmware, to operate the system of the invention.

The description of the operation of the data processing apparatus 340 is confined to that of a personal computer or PC 340 however it is not to be taken as a limitation of the invention. For instance, base station 2 may be provided by a smartphone which is (i) programmable, (ii) contains a data store, (iii) a radio frequency interface (or multiple RF interfaces), and (iv) antenna. Indeed, for certain environments and uses, a smartphone can be used as both the active tag (with inbuilt sensors—eg accelerometers, temperature and light sensors, and the provision to hook up external sensors eg. headphones and other sensors such as ECG sensors via the smartphone expansion ports) and where the base station could be effectively a server on the internet connected by way of internet connection with the smartphone.

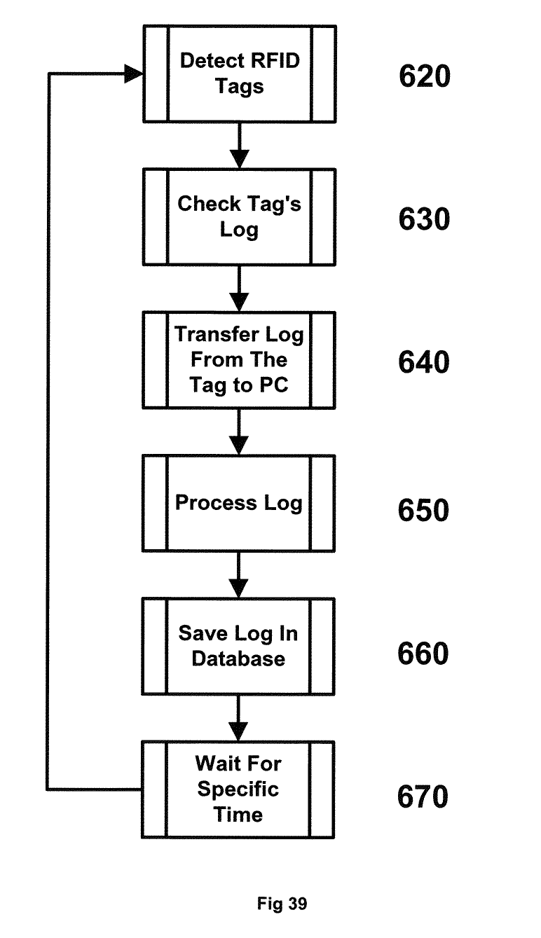

A PC 340 communicating with the base station transceiver 320 has implemented in its software, modules/method depicted in FIG. 39 for communicating with active tags 10 (or 15 or 540) and its database. The PC 340 may communicate with the transceiver 320 through a USB or other port. It also can use the serial port for communicating with the base station antenna (not shown). The PC 340 may send commands to the base station antenna 20, via the transceiver 340, to control it. This control can be in order to search for active tags 10 (or 15 or 540) in the field by way of unique identification data presented by the active tag 10 to the PC 340, or transferring sensor data from tags. The PC 340 may be programmed to use a timer 670 event to search for tags in the field. The timer may check the tag's logged data 630 and transfer 640 them to the PC 340 and save them.

The data transferred from the tag to the PC 340 may be encrypted and compressed for increased security and increased data transfer rate. Only an authorised PC 340 can decode and decompress the data. This may be via a password. The data may have time stamps or consist of start up time and logging time interval to calculate the time each sample was taken. This information is saved in the database along with the data sample.

The PC 340 provides the ability to start or stop a search for tags and communicate with them as set out on FIG. 39. The PC 340 searches for active tags 10 (or 15 or 540) in the field and as soon as it finds an active tag 620, it retrieves the active tag's status 630 to find out the amount of data stored in it. If the amount of data stored in the tag reaches or exceeds the threshold set in software, then the software starts reading the data 640 from tag's log. The software may process 650 the received data as soon as each packet is received successfully. The processed data can be saved in database 660. The PC 340 prompts the user to save any newly detected tags in a database 660. The PC 340 can communicate with one or more tags 620 at a time or in succession and read their logged data. The PC 340 may display the logged data using a chart or control. The data might be shown as parameter value against time of sampling. The PC 340 allows the user to choose a specified tag from a list of detected tags in the field and display its data in a chart. The PC 340 may provide the ability to zoom the view of the data chart using a specified date and time or by input from a touch pad or mouse attached to the PC 340.

The data received from the active tags 10 (or 15 or 540) can be kept in the database for future use or for keeping a history of wearer's data. The data may also contain records of the wearers medical and service records which may assist in any decision making concerning the treatment or future duties of the wearer that would affect the wearers survivability.

The software on PC 340 can process the monitoring parameters received from the tag and display the possible risks that were threatening the user. This may be done in immediately after scanning such that a person being scanned can have an indication such as a visual alert or audible alert that indicates to the wearer that hazardous conditions were experienced. Indeed, a visual indication indicating that a tag has been read may be provided in doorways and at security checkpoints in which the wearer is not permitted to continue unless the tag has been read, and if it is read and indicates hazardous conditions were experienced, the wearer is diverted from the progressing stream of personnel.

In embodiments of the invention for use other than in the armed forces, the active tag 10 itself may also comprise LED indicators or other feedback mechanisms (for example audible signals, text messages and other communication modes) to indicate various statuses including whether the wearer has been subject to any toxic chemicals or is otherwise in a state where medical assistance is required or advised, outside of the range of a base station.

The person skilled in the art will appreciate from the foregoing that the methods described for utilising the active uniform 1 and base station 2 of the invention provide the means for measuring the wearers environment and/or physiology. With this information a wearer is able to enhance its survivability in hazardous conditions when that information is made known the wearer and/or OHS and/or medical professionals.

It will also be apparent to persons skilled in the art that various modifications may be made in details of design and construction of the active uniform system 10 described above without departing from the scope or ambit of the present invention.

### INDUSTRIAL APPLICABILITY

Active uniforms that enhance the survivability of the wearer would find application in military, industrial, medical and civil applications where the wearer is subject to hazardous conditions or is otherwise the subject of monitoring for signs of ill health

1-39. (canceled)

40. A system for sensing, logging and presenting physiological and/or environmental conditions of a wearer of an item of active uniform, the system comprising an active uniform and a base station;
the active uniform comprising:
(i) an item of uniform;
(ii) at least one electronic sensor for sensing an aspect of the wearer's environment or of the wearer's physiology; and
(iii) an active tag mounted on the item of uniform for communicating with the at least one electronic sensor, wherein the active tag comprises;
a. sensor interface for interfacing the at least one electronic sensor to the active tag;
b. at least one microcontroller adapted to:
process the signals received over the sensor interface from the at least one electronic sensor into sensor data;
send and receive sensor data, and identification data which identifies the wearer, to a base station transceiver; and
send/receive and/or process instructions sent or received from the base station transceiver, including where necessary, the control of other components of the active tag;
c. data store for recording and storing, at least, sensor data;
d. radio frequency interface adapted to convert electrical signals output by the microcontroller into radio signals adapted to be received by the base station transceiver and to convert radio signals received from the base station transceiver, into electrical signals that are adapted to be received by the microcontroller;
e. at least one tag antenna for transmitting radio signals to and from the base station transceiver;
f. power supply and regulation means;
the base station comprising:
(i) at least one base station antenna;
a data processing apparatus connected to the base station transceiver, which is adapted to, at least, receive and store data transmitted by the active tag of the active uniform, including at least, sensor and identification data; and
(ii) wherein the at least one electronic sensor and active tag are waterproof and wherein the active uniform can be laundered in conventional laundry machines without removing the at least one electronic sensor and active tag.

41. The system of claim 40 wherein the power supply and regulation means of the active tag comprises an internal power source, including a battery, connected to a power management unit in turn connected to, and controlled by, the active tag microcontroller.

42. The system of claim 41 wherein, at least, the sensor interface, microcontroller, data store, battery and power regulator are housed in a waterproof, chemically resistant and tamper resistant container.

43. The system of claim 42 in which the at least one electronic sensor is selected from the group comprising:
(i) GPS and/or accelerometers for determining the location and/or motion of the wearer;
(ii) electronic sensors for determining the respiration of the wearer;
(iii) electronic sensors for determining the blood pressure of the wearer;
(iv) electronic sensors for determining the blood glucose levels of the wearer;
(v) electronic sensors for measuring the brain activity of the wearer;
(vi) electronic sensors for determining the impacts or forces imparted on the wearer;
(vii) electronic sensors for determining the exposure to chemical and/or biological agents by the wearer;
(viii) electronic sensors for determining radiation dosage to the wearer;
(ix) electronic sensors for determining the ambient temperature, humidity or barometric pressure;
(x) electronic sensors for determining the number of wash cycles the uniform has been subjected to;
(xi) electronic sensors for determining the degree of fading of the uniform material.

44. The system of claim 43 wherein there are a plurality of different electronic sensors connected to the sensor interface and wherein at least one sensor is located outside of the container and wherein the at least one sensor located outside of the container is connected to the sensor interface inside the container, by way of a washable electrical conductor.

45. The system of claim 43 wherein there are a plurality of different electronic sensors connected to the sensor interface and wherein at least one sensor is located inside of the container.

46. The system of claim 45 wherein there are at least two different electronic sensors located outside of the container comprising at least an ECG sensor, and a temperature sensor and at least one electronic sensor located within the container comprising an accelerometer.

47. The system of claim 46 wherein the ECG sensor has electrodes that form part of the fabric of the uniform worn adjacent to the wearer's skin and wherein there is provided a strap, belt or other tensioning means for maintaining the electrodes against the surface of the wearer's skin, and wherein the electrodes are flexible and arranged in a stretchable configuration so as to permit free movement by the wearer.

48. The system of claim 45, wherein there are pressure and blast sensors which are connected to the sensor interface of the active tag, and housed within the waterproof, chemically resistant and tamper resistant container.

49. The system of claim 45 wherein the microcontroller combines and processes sensor data from the plurality of different electronic sensors to arrive at a measure of something not able to be measured directly or is difficult to measure directly, by way of an electronic sensor.

50. The system of claim 49 wherein temperature, heart rate and movement data are combined and processed to arrive at a measure of the wearer's stress level.

51. The system of claim 45 wherein the container further comprises, means for detecting tampering with the circuitry contained within the container, including the provision of a conductive tamper track which forms a circuit that is readable by the microcontroller, where the conductive tamper track is provided in elements that surround the electrical components desired to be monitored, such that any attempts at accessing the electrical components would break the conductive track, result in the circuit being broken, and the identification of the same by the microcontroller.

52. The system of claim 51 in which the container further contains a capacitor, whereupon the detection of tampering the capacitor in stored energy erases the contents of the data store and any stored firmware or data contained within the memory of the microcontroller, even if the battery has been disconnected.

53. The system of claim 45 wherein the data processing apparatus of the base station is a standalone device capable of receiving and storing data from active uniforms and is further adapted to communicate the stored data when a suitable data connection is available.

54. The system of claim 53 wherein the data processing apparatus is adapted to identify wearers whose exposure to certain hazardous activities or risks exceed programmable risk thresholds.

55. The system of claim 54 wherein the identification of wearers whose exposure to certain hazardous activities or risks exceed programmable risk thresholds includes providing visual, audible, or textual alerts, either directly via the active tag, or alternatively, by reference to some aspect of the base station apparatus.

56. There is provided a method of enhancing the survivability of the wearer of at least one item of active uniform, the method comprising:
providing at least one item of active uniform which continuously monitors and records the environment and/or physiological functions of the wearer's data;
transmitting the data to an authenticated transceiver;
receiving the data at the authenticated transceiver and communicating the data to a data processing device; and
processing the data to determine whether either the environmental conditions experienced by the wearer or the wearer's own physiological data indicates that the wearer should be taken out from active service, or have future duties modified, or medically treated, so as to enhance the survivability of the wearer.

57. The method of claim 56 where the step of receiving the data involves the wearer passing though a zone of radio communication located at doorways, hatches or passageways wherein the wearer is not permitted to proceed through the zone, until the tag has been read, where the reading of the tag is indicated by way of a visual indicator.
